(19) United States
(12) Patent Application Publication
(10) Pub. No.: US 2005/0049538 A1
(43) Pub. Date: Mar. 3, 2005

(54) Stick containing a product which the user can bring into contact with an absorbent material

(76) Inventor: Nicolas Trevillot, La Varenne Saint Hilaire (FR)

Correspondence Address: YOUNG & THOMPSON, 745 SOUTH 23RD STREET, 2ND FLOOR, ARLINGTON, VA, 22202, US

(21) Appl. No.: 10/494,287

(22) PCT Filed: Oct. 29, 2002
(86) PCT No.: PCT/FR02/03718

(30) Foreign Application Priority Data
Nov. 2, 2001 (FR) 01/14232

(51) Int. Cl. A61M 35/00

(52) U.S. Cl. 604/2

(57) ABSTRACT

The invention concerns a stick containing a liquid, pasty or gelled product (6) which the user can bring into contact with an absorbent material located at one end of said stick, comprising: a first stem (1), a second hollow stem (4) containing said product, (6), whereof one end is provided with an absorbent material (5; 11) and the other end receives said first stem (1); said first stem (1) capable of sliding inside said second stem (4) so as to push said product (6) towards said absorbent material (5; 11); said second stem (4) comprising at its end provided with an absorbent material (5; 11) closure means for preventing contact between the product (6) and the absorbent material (5; 11) before the stick is used. The invention is characterized in that it comprises means for rupturing said closure actuated by sliding the first stem (1) in the second stem (4).

The invention relates to the field of packaging of products in the liquid, paste-like or gel-like state, and the release thereof onto an absorbent material in order to make them accessible to a consumer, for example, for the application thereof to a surface.

Padded sticks, generally known as “cotton buds”, are often used in daily life for cleaning body parts, such as the auditory canals, or various objects. It is often necessary or advantageous to coat the surface to be cleaned with a product in the liquid, paste-like or gel-like state, such as an antiseptic solution. To this end, it is possible to soak a padded end of the stick in a container containing the product which is to be applied to the zone to be treated.

Therefore, conventional sticks require, for these methods of use, that the consumer has at his disposal, in addition to the stick, a separate container containing a relatively large quantity of the product, even if the quantity to be applied must be minimal. This situation is often inconvenient owing to the multiple operations which the user has to carry out; in particular, there is always a risk of overturning the container.

Padded sticks are known and are described, for example, in documents WO-A-00/00147 or WO-A-98/11852, wherein the stick is hollow and contains liquid, such as an antiseptic solution, an eye lotion, etc. This liquid can be expelled towards one end of the stick in order to impregnate the padded portion therewith by means of a pressure applied manually by the user. A portion of the stick can, to this end, have an enlargement which acts as a liquid reservoir which increases the capacity of the stick and a preferential zone for the application of the expulsion pressure. The other end of the stick is not provided with an absorbent material, or it is not in communication with the interior of the stick and remains dry. This second configuration allows the user to precede or follow the wet cleaning phase or liquid application phase with a dry cleaning phase or a phase for absorbing excessively deposited liquid.

These devices have the following disadvantages. Firstly, the application of a pressure to the stick is a difficult operation to gauge. An excessively weak pressure risks the expulsion of an insufficient quantity of product in the direction of the absorbent material. Conversely, an excessively abrupt pressure can bring about a surge of product such that it risks being partially ejected from the stick instead of slowly impregnating the absorbent material. The dimensional and mechanical features of the tube of the stick must be selected with care in order to limit these disadvantages and, in any case, it is not possible to prevent the user from carrying out the operation without sufficient attention.

Secondly, simple pressure applied at a given point of the tube does not generally allow complete expulsion of the product contained in the stick. Therefore, it is necessary for the user either to apply this pressure by displacing his clenched fingers along the tube in the direction of the absorbent material, which is inconvenient, in particular if the tube has great rigidity, or to resign himself to using only some of the product. The provision of a reservoir in the tube, as in WO-A-00/00147, allows the quantity of product that the user can expel by simple pressure to be increased. However, this solution complicates the manufacture of the stick and increases the spatial requirement thereof and even so does not in any case allow all of the product contained in the stick to be readily used.

Document U.S. Pat. No. 3,519,364 also discloses application devices in two pieces which slide one inside the other. A tubular piece is covered at one of the ends thereof by the absorbent material and contains the product to be applied. This piece is closed by a cover. The other piece is a rod which the user can slide inside the tubular piece in the manner of a piston in order to reduce the free internal volume of the tubular piece and, in this manner, to push the product towards the absorbent material, the cover being broken by the effect of the pressure of the product alone.

In this last type of device, one disadvantage is that the expulsion of the product is dependent on the cover being broken by the effect of the pressure of the product alone. During the sliding of the rod, therefore, the user experiences “resistance” at the moment when the product applies his effort to the cover, without the cover being broken. The user must then apply an effort greater than that which was previously necessary in order to cause the rod to advance in the tube in the absence of compression of the product, and greater than that which will then be necessary in order to expel the product completely. This “resistance” effect is unpleasant and can also lead to an excessively violent expulsion of the product out of the tube in the moments following the breaking of the cover, if the user does not instantly reduce the effort which he is applying to the rod.

The object of the invention is to provide a stick of the above-described type which is free from this disadvantage. To this end, the invention relates to a stick which contains a product in the liquid, paste-like or gel-like state which the user can bring into contact with an absorbent material located at one end of the stick, of the type comprising:
a first solid or hollow rod which is provided, if it is hollow, with a closure device at one of the ends thereof;
a second hollow rod which contains the product, one end of which is provided with an absorbent material and the other end of which receives the first rod by means of that end thereof which may be provided with a closure device;
the first rod being able to slide inside the second rod in order to push the product towards the absorbent material by means of the end thereof;
the second rod comprising, at the end thereof provided with an absorbent material, closure means which prevent contact between the product and the absorbent material before the stick is used;
   characterised in that it comprises means for breaking the closure means which prevent contact between the product and the absorbent material, which means are controlled by the sliding of the first rod in the second rod.

The means which prevent contact between the product and the absorbent material can be constituted by a cover which covers the second rod.

The means for breaking the closure means can be constituted by a protuberance which is fixedly joined to the first rod. The means which prevent contact between the product and the absorbent material can be constituted by a wall which delimits the end portion of the second rod and which has a frustoconical form which tapers in the direction of the end thereof.

The wall preferably comprises lines of reduced resistance.

The end of the first rod can have a cross-section which is provided with branches which are intended to apply a pressure to the wall perpendicularly to the lines of reduced resistance.

That end of the first rod which is not received in the second rod can be provided with an absorbent material.

The absorbent material which is located at one end of the second rod can be cotton wool.

The absorbent material which is located at that end of the first rod which is not received by the second rod can be cotton wool.

The absorbent material which is located at the end of the second rod can be a sponge-like material.

That end of the first rod which is not received in the second rod can comprise an enlargement and the second rod can comprise a ring on the outer wall thereof.

The first rod can also contain a product in the liquid, paste-like or gel-like state.

The products contained in the first rod and the second rod can be different.

At least one of the products can be an antiseptic product.

At least one of the products can be a product for cosmetic use.

At least one of the products can be a cleaning product.

At least one of the products can be a perfume.

At least one of the products can be an adhesive.

At least one of the products can be a paint.

As will be appreciated, the invention consists in the provision of means assisting the breaking of the cover or any other type of functionally equivalent wall which prevents contact between the product and the material used for the application thereof when the stick is in the unused state. The expulsion of the product is thus no longer dependent only on the pressure applied to the wall by the product. The breaking of the wall is brought about in this manner with greater reliability. In particular, it can be brought about without the user perceiving the advance of the rod to be braked too substantially, which increases the comfort of use and can make the expulsion of the product more progressive.

The uses of these sticks can be very varied and will depend on the natures of the product(s) which they contain and the absorbent material which the product impregnates. They can relate to fields other than the application of medicinal or cosmetic products.

The invention will be better understood from a reading of the description below which is given with reference to the appended Figures, in which:

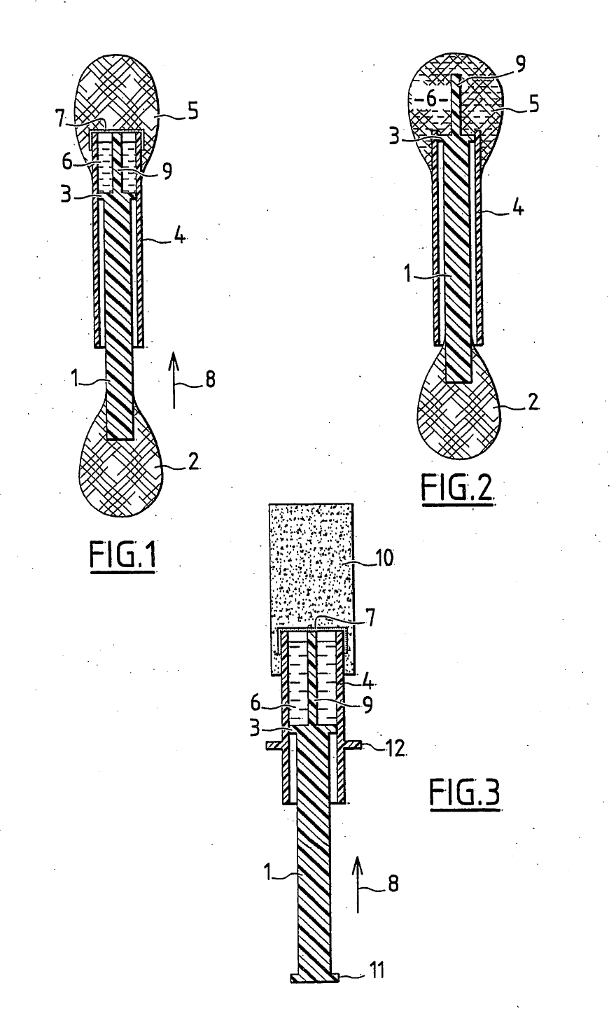

FIG. 1 is a sectioned view of an embodiment of the padded stick according to the invention before use;

FIG. 2 shows the same stick ready for use, one of the padded ends being impregnated with the product which is initially contained by the stick;

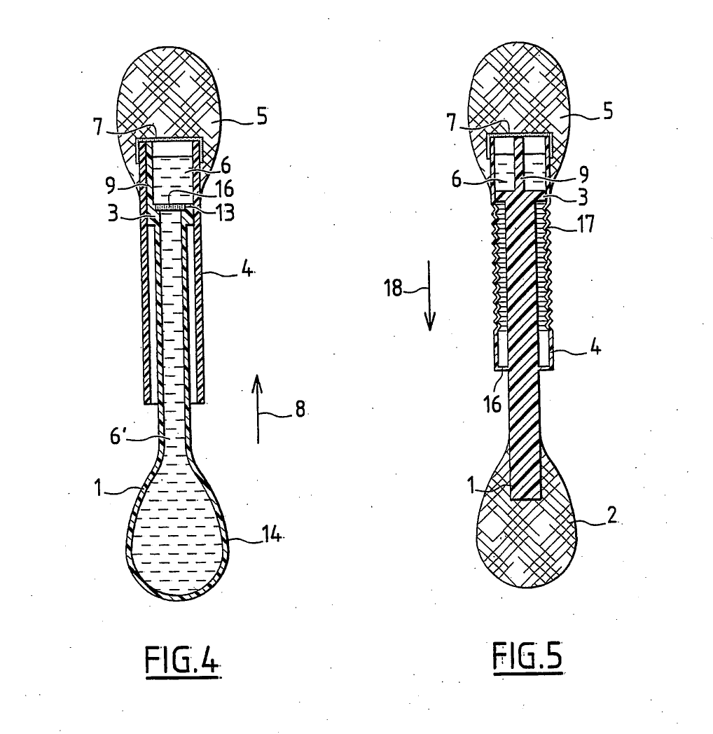

FIG. 3 is a sectioned view of a variant of the invention using one end of the stick which comprises an absorbent sponge-like material;

FIG. 4 is a sectioned view of another variant of the invention, in which the two rods of the stick contain a product to be applied;

FIG. 5 is a sectioned view of another variant of the invention, in which the sliding of one rod of the stick in the other is carried out by one of the rods being folded up on itself;

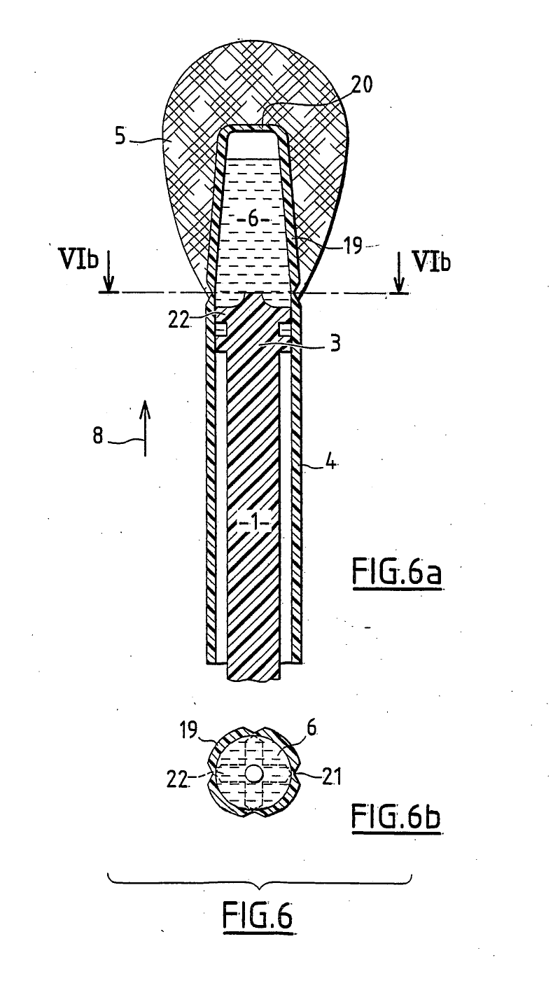

FIG. 6 is a longitudinal section (FIG. 6a) and a cross-section along VIb-VIb (FIG. 6b) of another embodiment of the stick according to the invention.

A first embodiment of the invention is illustrated in FIG. 1. This is a stick comprising a wad of cotton wool at the two ends thereof, of the type conventionally used, for example, for cleaning auditory canals.

The stick comprises a first generally cylindrical rod 1 of a material such as polypropylene. This first rod 1 comprises, at one of the ends thereof, a first cotton wool wad 2 which is fixed to the outer wall thereof by means conventionally used for this purpose. At the other end thereof, the first rod 1 comprises an enlargement 3 which slightly increases the diameter thereof. This enlargement 3 can, as illustrated, be structurally integrated in the first rod 1, which is then solid, or be constituted by a cap which is initially independent of the first rod 1. In this latter case, the first rod 1 can be hollow, the cap then also acting as a device for closing the rod 1. The stick comprises a second rod 4 which must be hollow. One of the ends thereof is surrounded by a second cotton wool wad 5. The other end receives the enlargement 3 of the first rod 1. The diameter of the enlargement 3 and the inside diameter of the second rod 4 are selected so that they allow the first rod 1 to slide inside the second rod 4, whilst maintaining leak-tight mutual contact. This leak-tightness is necessary because the second rod 2 contains a product 6 in the liquid, paste-like or gel-like state, which is intended to impregnate the second cotton wool wad 5 during use of the stick. In order to maintain this leak-tightness at the other end of the stick before it is used, and also to prevent contact between the product 6 and ambient air, if necessary, a cover 7 covers the second rod 4 so as to close it. Depending on the envisaged uses, the product 6 can be a product intended to assist in the cleaning of the auditory canal by dissolving cerumen, or an antiseptic solution or any other liquid, paste-like or gel-like product for medical or cosmetic use by local application to the body. The product 6 can also be a cleaning solution or a different substance for non-medical use, whose application can advantageously be carried out by means of the cotton wool wad 5.

According to the invention, the first rod 1 comprises a protuberance 9 at the end thereof that is introduced into the second rod 4. The protuberance 9 has a length which allows it to come into contact with the cover 7 during the sliding of the first rod 1 before the product 6 fills all of the space available between the enlargement 3 and the cover 7, or at the latest at this precise moment.

The stick is used as follows. The user applies a pushing action to the first rod 1 according to the arrow 8 so as to cause the first rod 1 to be introduced more deeply inside the second rod 4 in the manner of a piston. Under the effect of the pressure applied by the protuberance 9, the cover 7 breaks and the product 6 impregnates the second cotton wool wad 5. FIG. 2 shows the stick after complete introduction of the first rod 1 into the second rod 4 which has brought about complete expulsion of the product 6 (the broken cover 7 has not been illustrated in FIG. 2). The product 6 can then be applied by means of the second cotton wool wad 5 to the zone to be treated.

The first cotton wool wad 2, which has remained dry, can be used to remove a surplus of product 6 which has been spread over the treated zone. It can also be used before the second wad 5 in order to carry out preliminary dry cleaning of the zone to be treated.

The material of the rod 4 must be impermeable to this product 6. In this case too, polypropylene can be used for a number of applications. However, other materials which have leak-tightness relative to the product 6 and suitable mechanical characteristics can be used.

The stick according to the invention has in particular the following advantages over sticks which contain a product of the previously known types.

The expulsion of the liquid is carried out by means of a translation movement, which is simpler to control by the user than the usual pressing movement in the sticks of WO-A-00/00147 or WO-A-98/11852. Furthermore, this translation movement, when it has been carried out until the first rod 1 is completely introduced into the second rod 4, allows the complete expulsion of the product 6 out of the second rod 4. The user has no difficulty in carrying out and verifying this complete expulsion because it is simply necessary for this purpose for him to control the depression of the first rod 1. It is even possible for him, if he wishes, to expel only some of the product 6 during at least the first use of the stick. Graduations marked on the first rod 1 can give an indication regarding the quantity of product 6 which has effectively been expelled. So that such a method of use is possible, it is naturally necessary for the product 6 to have a consistency which allows it to remain in the stick, even after the cover 7 which may be present has been broken. It is also necessary for the properties thereof not to be affected too quickly by the possibility of exposure to air brought about by the cover 7 being broken. For this reason, such partial use cannot be advised if, for example, the product 6 must have aseptic properties when it is used.

Furthermore, the comfort and the ease of expulsion of the product 6 are further increased in comparison with the sticks of U.S. Pat. No. 3,519,364. The concentration of the effort applied to the cover 7 within the contact zone between the cover 7 and the protuberance 9, and not over the entirety of the surface of the cover 7, makes breaking the cover 7 easier because it requires less effort. Therefore, the user does not have to substantially increase the force which he applies to the rod 1 when the breaking of the cover 7 has to be brought about. The advance of the rod 1 is therefore effected without any surge and the expulsion of the product 6 can readily be controlled by the user.

Raised or knurled portions provided on the surfaces of the rods 1, 4 can facilitate the holding and handling of the stick by the user.

The variant of the invention illustrated in FIG. 3 differs from the preceding one in that:
the cotton wool wad 2 of the first rod 1 has been dispensed with; therefore, the first rod 1 does not comprise any absorbent material;
and the cotton wool wad 5 of the second rod 4 has been replaced with a sponge-like material 10, which can be provided with a shape and dimensions which are well-defined, if necessary.

This last feature is particularly suitable when it is desirable to apply the product 6 to a small well-delimited surface. This can be the case for a given number of uses, whether medical or non-medical. The product 6 can be, for example, a paint, nail varnish, an adhesive.

An enlargement 11 provided at the free end of the first rod 1 and a ring 12 provided around the second rod 4 can, as illustrated, facilitate the holding and the use of the device, in the manner of a syringe. In this manner, the user can carry out, with one hand and in a simultaneous manner, the operations for impregnating the sponge-like material 10 and applying the product 6 to the surface to be treated.

Another conceivable use for the device can be the packaging of perfume samples, for which various types of absorbent materials can be used.

In place of a cotton wool wad 5 or a sponge-like material 10, it is possible to use in general any material which can temporarily retain the product 6, for example, in order to apply it to a surface.

By way of a variant, if the first rod 1 is hollow, it is possible to fill it with a product 6′ in order to constitute an additional reserve of the product 6 contained in the second rod 4. FIG. 4 shows such a variant. The first rod 1 is closed by a cover 13 at the end thereof that is engaged in the second rod 4 and comprises, at the other end thereof, an enlargement 14 which forms a reservoir. In this case, the protuberance 9 is positioned at the periphery of the enlargement 3 of the first rod 1 and not at the centre thereof, so as to allow the presence of the cover 13. In this manner, after the product 6 has been completely expelled from the second rod 4, as explained above, the user can expel the product 6′ which is located in the first rod 1 by pressing on the enlargement 15, as is known in the prior art. The cover 13 of the first rod 1 must not be damaged during the first phase of use of the stick so that the first rod 1 can act as a piston on the product 6 contained in the second rod 4. It is preferable for the cover 13 of the first rod 1 not to come into contact with the internal wall of the second rod 4 during the depression operation. To this end, the cover 13 can be adhesively bonded in a housing 16 which is provided on the upper surface of the enlargement 3, which also has the advantage of leaving space available for the arrangement of the protuberance 9.

By way of a variant, other means for expelling the product 6′ out of the first rod 1 could be envisaged. They could optionally allow expulsion of the product which is contained in the first rod 1 at the end thereof opposite that which is introduced into the second rod 4. It is also conceivable for the two rods 1, 4 each to contain a different product, the products 6, 6′ having to be or being able to be used successively in order, for example, to be mixed at the zone of application thereof after being stored separately until the stick is used.

The variant of the invention illustrated in FIG. 5 differs from the preceding variants in that the sliding of the first rod 1 inside the second rod 4 is effected in the following manner. The end 16 of the second rod 4 opposite the end having the cotton wool is fixedly joined to the first rod, preferably in a leak-tight manner. At least a portion 17 of the length of the second rod 4 has a given flexibility and is formed in an accordion-like manner in order to be able to be folded up on itself when the user applies an effort, which is directed according to the arrow 18, to the second rod 4, tending to bring together the two ends of the second rod 4. This effort and this folding of the second rod 4 on itself have the effect of reducing the volume available for the product 6 in the second rod 4 owing to the enlargement 3 and that end of the second rod 4 which carries the second cotton wool wad 5 being brought together and, in this manner, of allowing the product 6 to be expelled after the cover 7 has been broken by the protuberance 9.

It will be appreciated that the various configurations of the device according to the invention which have been described are merely examples whose different features can be replaced with functionally equivalent features. In particular, the presence of the enlargement 3 at the end of the first rod 1 is not strictly necessary if the dimensional features of the rods 1 and 4 and the consistency of the product 6 allow a leak-tightness of the stick to be obtained which is judged to be sufficient, in the state in which it is not in use and during use.

Another variant of the invention is illustrated in FIG. 6. In this variant, the means for closing the second rod 4 are no longer constituted by a cover, but instead by a wall 19 which is integral with the remainder of the second rod 4. This wall 19 delimits the end portion of the second rod 4 which contains the product 6 and confers on it a frustoconical form which tapers in the direction of the end 20 thereof. Lines of reduced resistance are preferably provided in the wall 19, for example, in the form of longitudinal grooves 21 which meet in the region of the end 20 of the wall 19. In this manner, when the user depresses the first rod 1 into the second rod 4, the enlargement 3 applies a force to the wall 19, which force tends to widen it, and the wall 19 breaks in this manner along the grooves 21, if they are present, allowing the product 6 which is intended to impregnate the cotton wool wad 5 to be discharged.

As illustrated in FIG. 6b, the upper surface of the enlargement 3 preferably has a cross-section which is provided with lateral branches 22 which apply, in a preferential manner, a pressure to the wall 19 at right-angles to the grooves 21 during the advance of the first rod 1.

This variant, unlike the preceding variant, does not cause the introduction of a protuberance of the first rod 1 without any protection at the centre of the cotton wool wad 5, which can constitute an advantage in some applications, for example, for cleaning auditory canals, where the protuberance could be in a position to injure the user if it were to be exposed during the operation.

In this variant, the liquid 6 can fill all of the available space in the stick in the unused state, if the piston brings about, from the start of the movement thereof, an increase in the volume defined by the wall 19. This achieves good use of the available space.

In all of the examples of use of the invention which have been described, it is preferable for the product 6 not to fill completely all of the space available between the two rods 1, 4. In this manner, it is ensured that the efforts which tend to cause the end of the second rod 4 (or the cover 7 or the wall 19) to break are applied by the enlargement 3 of the first rod 1 or a protuberance 9 which it carries,

and not by the product 6 itself. The introduction of the product 6 into the stick must preferably be carried out in an inert atmosphere or under vacuum if the product 6 can be degraded upon contact with air which would otherwise be able to be captured therewith in the stick.

The person skilled in the art will also readily be able to combine features taken from the various configurations described. Finally, it will be appreciated that the examples of use of the device according to the invention which have been set out are in no way limiting.

1. Stick containing a product (6) in the liquid, paste-like or gel-like state which the user can bring into contact with an absorbent material located at one end of the stick, of the type comprising:
a first solid or hollow rod (1) which is provided, if it is hollow, with a closure device at one of the ends thereof;
a second hollow rod (4) which contains the product (6), one end of which is provided with an absorbent material (5; 11) and the other end of which receives the first rod (1) by means of that end thereof which may be provided with a closure device;
the first rod (1) being able to slide inside the second rod (4) in order to push the product (6) towards the absorbent material (5; 11) by means of the end (3) thereof;
the second rod (4) comprising, at the end thereof provided with an absorbent material (5; 11), closure means which prevent contact between the product (6) and the absorbent material (5; 11) before the stick is used;
characterised in that it comprises means for breaking the closure means which prevent contact between the product (6) and the absorbent material (5; 11), which means are controlled by the sliding of the first rod (1) in the second rod (4).

2. Stick according to claim 1, characterised in that the means which prevent contact between the product (6) and the absorbent material (5; 11) are constituted by a cover (7) which covers the second rod (4).

3. Stick according to claim 1, characterised in that the means for breaking the closure means are constituted by a protuberance (9) which is fixedly joined to the first rod (1).

4. Stick according to claim 1, characterised in that the means which prevent contact between the product (6) and the absorbent material (5; 11) are constituted by a wall (19) which delimits the end portion of the second rod (4) and which has a frustoconical form which tapers in the direction of the end (20) thereof.

5. Stick according to claim 4, characterised in that the wall (19) comprises lines of reduced resistance (21).

6. Stick according to claim 5, characterised in that the end (3) of the first rod has a cross-section which is provided with branches (22) which are intended to apply a pressure to the wall (19) perpendicularly to the lines of reduced resistance (21).

7. Stick according to claim 1, characterised in that that end of the first rod (1) which is not received in the second rod (4) is provided with an absorbent material (2).

8. Stick according to claim 7, characterised in that the absorbent material (5) which is located at one end of the second rod (4) is cotton wool.

9. Stick according to claim 8, characterised in that the absorbent material (2) which is located at that end of the first rod (2) which is not received by the second rod (4) is cotton wool.

10. Stick according to claim 1, characterised in that the absorbent material (11) which is located at the end of the second rod (4) is a sponge-like material.

11. Stick according to claim 1, characterised in that that end of the first rod (1) which is not received in the second rod (4) comprises an enlargement (12) and in that the second rod (4) comprises a ring (13) on the outer wall thereof.

12. Stick according to claim 1, characterised in that the first rod (1) also contains a product (6′) in the liquid, paste-like or gel-like state.

13. Stick according to claim 12, characterised in that the products (6, 6′) contained in the first rod (1) and the second rod (4) are different.

14. Stick according to claim 1, characterised in that at least one of the products (6, 6′) is an antiseptic product.

15. Stick according to claim 1, characterised in that at least one of the products (6, 6′) is a product for cosmetic use.

16. Stick according to claim 1, characterised in that at least one of the products (6, 6′) is a cleaning product.

17. Stick according to claim 1, characterised in that at least one of the products (6, 6′) is a perfume.

18. Stick according to claim 10, characterised in that at least one of the products (6, 6′) is an adhesive.

19. Stick according to claim 10, characterised in that at least one of the products (6, 6′) is a paint.

20. Stick according to claim 1, characterised in that the absorbent material (5) which is located at one end of the second rod (4) is cotton wool.
